(19) United States
(12) Patent Application Publication
(10) Pub. No.: US 2011/0264144 A1
(43) Pub. Date: Oct. 27, 2011

(54) LUMBAR-SACRAL STRUT

(75) Inventors: Amy L. Arthur, Mountain View, CA (US); Philippa E.K. Gray, Sunnyvale, CA (US); Mark B. Quinto, San Jose, CA (US)

(73) Assignee: KYPHON SARL, Neuchatel (CH)

(21) Appl. No.: 12/766,642

(22) Filed: Apr. 23, 2010

(51) Int. Cl. A61B 17/70

(52) U.S. Cl. 606/278; 606/246

(57) ABSTRACT

Medical devices for the treatment of spinal conditions are described herein. The medical device includes a main body that is adapted to be placed between the L5 vertebra and the sacrum so that the main body acts as a spacer with respect to the L5 vertebra and the sacrum to maintain distraction therebetween when the spine moves to extension.

### BACKGROUND

This invention relates generally to the treatment of spinal conditions, and more particularly, to the treatment of spinal stenosis using devices for implantation between adjacent spinous processes.

The clinical syndrome of neurogenic intermittent claudication due to lumbar spinal stenosis is a frequent source of pain in the lower back and extremities, leading to impaired walking, and causing other forms of disability in the elderly. Although the incidence and prevalence of symptomatic lumbar spinal stenosis have not been established, this condition is the most frequent indication of spinal surgery in patients older than 65 years of age.

Lumbar spinal stenosis is a condition of the spine characterized by a narrowing of the lumbar spinal canal. With spinal stenosis, the spinal canal narrows and pinches the spinal cord and nerves, causing pain in the back and legs. It is estimated that approximately 5 in 10,000 people develop lumbar spinal stenosis each year. For patients who seek the aid of a physician for back pain, approximately 12%-15% are diagnosed as having lumbar spinal stenosis.

Common treatments for lumbar spinal stenosis include physical therapy (including changes in posture), medication, and occasionally surgery. Changes in posture and physical therapy may be effective in flexing the spine to decompress and enlarge the space available to the spinal cord and nerves—thus relieving pressure on pinched nerves. Medications such as NSAIDS and other anti-inflammatory medications are often used to alleviate pain, although they are not typically effective at addressing spinal compression, which is the cause of the pain.

Surgical treatments are more aggressive than medication or physical therapy, and in appropriate cases surgery may be the best way to achieve lessening of the symptoms of lumbar spinal stenosis. The principal goal of surgery is to decompress the central spinal canal and the neural foramina, creating more space and eliminating pressure on the spinal nerve roots. The most common surgery for treatment of lumbar spinal stenosis is direct decompression via a laminectomy and partial facetectomy. In this procedure, the patient is given a general anesthesia as an incision is made in the patient to access the spine. The lamina of one or more vertebrae is removed to create more space for the nerves. The intervertebral disc may also be removed, and the adjacent vertebrae may be fused to strengthen the unstable segments. The success rate of decompressive laminectomy has been reported to be in excess of 65%. A significant reduction of the symptoms of lumbar spinal stenosis is also achieved in many of these cases.

Alternatively, the vertebrae can be distracted and an interspinous process device implanted between adjacent spinous processes of the adjacent vertebrae to maintain the desired separation between the vertebral segments. Such interspinous process devices typically work for their intended purposes. However, because of the human anatomy some of these devices are not readily usable between certain vertebrae.

The spine is divided into regions that include the cervical, thoracic, lumbar, and sacrococcygeal regions. The cervical region includes the top seven vertebrae indentified as C1-C7. The thoracic region includes the next twelve vertebrae identified as T1-T12. The lumbar region includes five vertebrae L1-L5. The sacrococcygeal region includes five fused vertebrae comprising the sacrum. These five fused vertebrae are identified as the S1-S5 vertebrae. Four or five rudimentary members form the coccyx.

The sacrum is shaped like an inverted triangle with the base at the top. The sacrum acts as a wedge between the two iliac bones of the pelvis and transmits the axial loading forces of the spine to the pelvis and lower extremities. The sacrum is rotated anteriorly with the superior endplate of the first sacral vertebra angled from about 30 degrees to about 60 degrees in the horizontal plane. The S1 vertebra includes a spinous process aligned along a ridge called the medial sacral crest. However, the spinous process on the S1 vertebrae may not be well defined, or may be non-existent, and therefore may not be adequate for supporting an interspinous process device positioned between the L5 and S1 spinous processes.

Thus, a need exists for a mechanism that will allow an interspinous process device to be readily positioned between the L5 and S1 spinous processes so that the L5 and S1 vertebrae can be distracted and the interspinous process device can maintain the desired separation between the vertebral segments.

### SUMMARY

The lumbar-sacral strut described herein includes a main body with a superior mounting portion and an inferior mounting portion formed along the superior and inferior portions of the main body. The superior mounting portion is adapted to engage an inferior portion of the lamina of the L5 vertebra. The inferior mounting portion is adapted to engage a superior portion of the sacrum. The lumbar-sacral strut is located along a portion of the spine lateral to the sagittal plane. Although a single lumbar-sacral strut may be used, two lumbar-sacral struts may be used with one such strut located on either side of the sagittal plane.

The superior and inferior mounting portions may take a variety of forms. For example, the superior mounting portion may indude a single superiorly extending flange adjacent to a superior shoulder similar to a ½ lap or rabbet joint. The inferior edge of the lamina of the L5 vertebra would thus abut the superiorly facing surface of the superior shoulder with the inferior portion of the lamina abutting the face of the superiorly extending flange. The inferior mounting portion may also take the form of a ½ lap or rabbet joint with a single inferiorly extending flange adjacent to an inferior shoulder. The superior edge of the sacrum would thus abut the inferiorly facing surface of the inferior shoulder with the superior portion of the sacrum abutting the face of the inferiorly extending flange. Alternatively, the superior mounting portion may include a pair of superiorly extending flanges with the superior shoulder extending therebetween. The inferior portion of the lamina of the L5 vertebra would thus fit between the two superiorly extending flanges with the inferior edge of the lamina resting on the superior shoulder. The inferior mounting portion may also include a pair of inferiorly extending flanges with an inferior shoulder extending therebetween. The superior portion of the sacrum would thus fit between the two inferiorly extending flanges with the superior edge of the sacrum abutting the inferior shoulder. The superior mounting portion may have a different mounting configuration than the inferior mounting portion.

Although the superior mounting portion and the inferior mounting portion formed in the lumbar-sacral strut are sufficient to prevent anterior or posterior migration of the device, fixation devices, such as a screw, may also be used to prevent lateral or medial migration of the lumbar-sacral strut. The strut may also be formed from so as to include a mechanism that allows the strut to be implanted in a collapsed configuration and then expanded along its longitudinal axis to an implanted, i.e., expanded configuration. The strut would thus provide distraction between the L5 vertebra and the S1 vertebra when in the expanded configuration. In addition, the connection between the multiple elements may be configured to allowed relative anterior or posterior motion between the elements.

### BRIEF DESCRIPTION OF THE DRAWINGS

FIG. 1 is a perspective view of one embodiment of a lumbar-sacral strut;

FIG. 2 is a perspective view of the other side of the lumbar-sacral strut shown in FIG. 1;

FIG. 3 is a perspective view of another embodiment of a lumbar-sacral strut;

FIG. 4 is a cross-sectional view of the lumbar-sacral strut shown in FIG. 3 taken along line 4-4 of FIG. 2;

FIG. 5 is a perspective view of another embodiment of a lumbar-sacral strut;

FIG. 6 is a posterior perspective view of a pair of lumbar-sacral struts, as shown in FIG. 1, mounted to the L5 vertebra and the sacrum;

FIG. 7 is a posterior view of the pair of lumbar-sacral struts, as shown in FIG. 1, mounted to the L5 vertebra and the sacrum;

FIG. 8 is a perspective view of another embodiment of a lumbar-sacral strut that allows the height of the strut to be compressed during implantation; and

FIG. 9 is a front perspective view of the embodiment of the lumbar-sacral strut shown in FIG. 8.

### DETAILED DESCRIPTION

As used in this specification and the appended claims, the singular forms “a,” “an” and “the” include plural referents unless the context clearly dictates otherwise. Thus, for example, the term “a member” is intended to mean a single member or a combination of members, and “a material” is intended to mean one or more materials, or a combination thereof. Furthermore, the words “proximal” and “distal” refer to directions closer to and away from, respectively, an operator (e.g., surgeon, physician, nurse, technician, etc.) who would insert the medical device into the patient, with the tip-end (i.e., distal end) of the device inserted inside a patient's body first. Thus, for example, the device end first inserted inside the patient's body would be the distal end of the device, while the device end last to enter the patient's body would be the proximal end of the device.

As used in this specification and the appended claims, the term “body” when used in connection with the location where the device of this invention is to be placed to treat lumbar spinal stenosis, or to teach or practice implantation methods for the device, means a mammalian body. For example, a body can be a patient's body, or a cadaver, or a portion of a patient's body or a portion of a cadaver.

As used in this specification and the appended claims, the term “parallel” describes a relationship, given normal manufacturing or measurement or similar tolerances, between two geometric constructions (e.g., two lines, two planes, a line and a plane, two curved surfaces, a line and a curved surface or the like) in which the two geometric constructions are substantially non-intersecting as they extend substantially to infinity. For example, as used herein, a line is said to be parallel to a curved surface when the line and the curved surface do not intersect as they extend to infinity. Similarly, when a planar surface (i.e., a two-dimensional surface) is said to be parallel to a line, every point along the line is spaced apart from the nearest portion of the surface by a substantially equal distance. Two geometric constructions are described herein as being “parallel” or “substantially parallel” to each other when they are nominally parallel to each other, such as for example, when they are parallel to each other within a tolerance. Such tolerances can include, for example, manufacturing tolerances, measurement tolerances or the like.

As used in this specification and the appended claims, the terms “normal”, “perpendicular” and “orthogonal” describe a relationship between two geometric constructions (e.g., two lines, two planes, a line and a plane, two curved surfaces, a line and a curved surface or the like) in which the two geometric constructions intersect at an angle of approximately 90 degrees within at least one plane. For example, as used herein, a line is said to be normal, perpendicular or orthogonal to a curved surface when the line and the curved surface intersect at an angle of approximately 90 degrees within a plane. Two geometric constructions are described herein as being “normal”, “perpendicular”, “orthogonal” or “substantially normal”, “substantially perpendicular”, “substantially orthogonal” to each other when they are nominally 90 degrees to each other, such as for example, when they are 90 degrees to each other within a tolerance. Such tolerances can include, for example, manufacturing tolerances, measurement tolerances or the like.

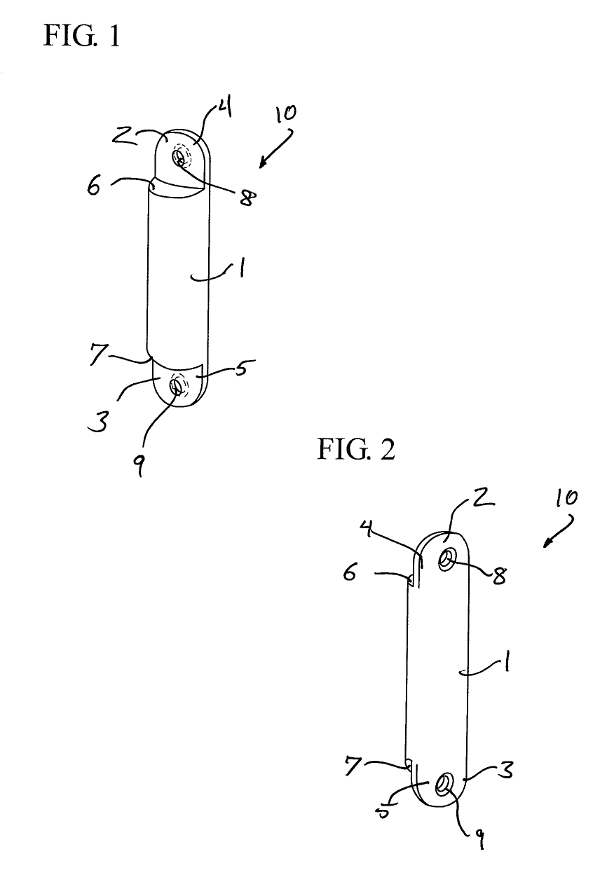

The lumbar-sacral strut 10 includes a main body 1 with a superior mounting portion 2 formed along a superior portion of main body 1 and an inferior mounting portion 3 formed along an inferior portion of main body 1. Superior mounting portion 2 is adapted to engage an inferior portion of the lamina of the L5 vertebra. Superior mounting portion 2 may take many configurations. For example, it may have a ½ lap, or rabbet, configuration. See FIGS. 1 and 2. As shown in these FIGS. superior mounting portion 2 includes a superior flange 4 which is adapted to abut an inferior portion of the lamina of the L5 vertebra. Superior mounting portion 2 also defines a superior shoulder 6, which is located along an inferior portion of superior flange 4 and has a superiorly facing surface that is adapted to abut the inferior edge of the lamina of the L5 vertebra. Similarly, inferior mounting portion 3 may also take many configurations, such as a ½ lap, or rabbet, configuration. As shown in FIGS. 1 and 2, inferior mounting portion 3 includes an inferior flange 5 which is adapted to abut a superior portion of the sacrum. Inferior mounting portion 3 also defines an inferior shoulder 7 which is located along a superior portion of inferior flange 5 and has an inferiorly facing surface that is adapted to abut the superior edge of the sacrum.

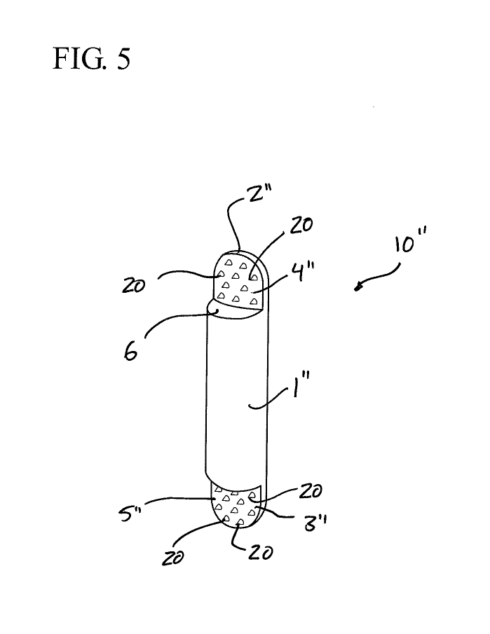

If desired, superior mounting portion 2 may include an opening 8 to allow a screw or other fastening device to extend therethrough to fix superior mounting portion 2 to the lower portion of the lamina of the L5 vertebra. And, inferior mounting portion 3 may also include an opening 9 to allow a screw or other fastening device to extend therethrough to fix inferior mounting portion 3 to the upper portion of the sacrum. Spikes, barbs or other similar elements 20 may be included on the face of superior flange 4″ of superior mounting portion 2″ that abuts the inferior portion of the lamina of the L5 vertebra and on the face of inferior flange 5″ of inferior mounting portion 3″ that abuts the superior portion of the sacrum. See FIG. 5. These elements 20 engage the bone of the lamina of the L5 vertebra and the sacrum respectively and, in combination with superior shoulder 6 and inferior shoulder 7 maintain lumbar-sacral strut 10″ in the desired position.

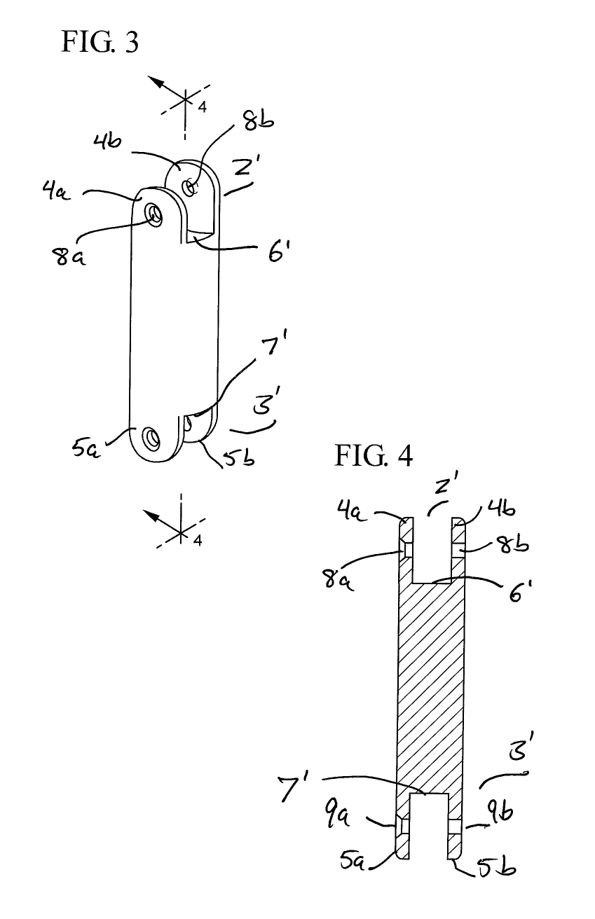

Alternatively, superior mounting portion 2′ may have a mortise-type configuration such that the inferior edge of the lamina acts as a tenon and can fit within the mortise of superior mounting portion 2′. Inferior mounting portion 3′ may have a similar configuration. See FIGS. 3 and 4. As shown in these FIGS., superior mounting portion 2′ includes two superior flanges 4a and 4b with a superior shoulder 6′ located therebetween. The space between superior flanges 4a and 4b is sized to allow the inferior portion of the lamina of the L5 vertebra to fit between superior flanges 4a and 4b and to rest on the superiorly facing surface of superior shoulder 6′. Although superior flanges 4a and 4b are shown as being parallel, they may also be oriented toward or away from each other to ensure a tight fit with the portion of the lamina that fits between the flanges. This arrangement fixes superior mounting portion 2′ to the inferior portion of the lamina of the L5 vertebra. In addition, if desired, a separate mounting element such as a screw can be used. As shown in FIGS. 3 and 4 superior flange 4a defines a hole 8a that extends through superior flange 4a and superior flange 4b defines a hole 8b that extends through superior flange 4b. These holes 8a and 8b allow a screw or other fastening device to pass through superior flanges 4a and 4b respectively and into the bone of the inferior portion of the lamina of the L5 vertebra. It is to be understood that these holes 8a and 8b and the aforementioned screw or other fastening device are not necessary for the operation of the lumbar-sacral strut described herein.

Inferior mounting portion 3′ may also have a mortise-type configuration. As shown in FIGS. 3 and 4, inferior mounting portion 3′ includes two inferior flanges 5a and 5b with an inferior shoulder 7′ located therebetween. The space between inferior flanges 5a and 5b is sized to allow the superior portion of the sacrum to fit between inferior flanges 5a and 5b and to abut the inferiorly facing surface of inferior shoulder 7′. Although inferior flanges 5a and 5b are shown as being parallel, they may also be oriented toward or away from each other to ensure a tight fit with the portion of the sacrum that fits between the flanges. This arrangement fixes inferior mounting portion 3′ to the superior portion of the sacrum. As with superior mounting portion 2′ shown in FIGS. 3 and 4, inferior flange 5a defines a hole 9a that extends through inferior flange 5a and inferior flange 5b defines a hole 9b that extends through inferior flange 5b. These holes 9a and 9b allow a screw or other fastening device to pass through inferior flanges 5a and 5b respectively and into the bone of the superior portion of the sacrum. It is to be understood that these holes 9a and 9b and the aforementioned screw or other fastening device are not necessary for the operation of the lumbar-sacral strut described herein.

It is not necessary for the superior mounting portion and the inferior mounting portion to have the same configuration. For example, the superior mounting portion may have a single flange, ½ lap, rabbet-type configuration and the inferior mounting portion could have a double flange, mortise-type configuration, or vice versa. In addition, it is not necessary for both the superior mounting portion and the inferior mounting portion to both include additional fixation devices. A single fixation device may be used solely in connection with the superior mounting portion or solely in connection with the inferior mounting portion. Although the configuration of the superior mounting portion and the inferior mounting portion are sufficient to prevent anterior or posterior migration of the lumbar-sacral strut, additional fixation devices, such as spikes, barbs or other elements or a screw or other fastening device may be used if desired to prevent lateral or medial migration of the lumbar-sacral strut.

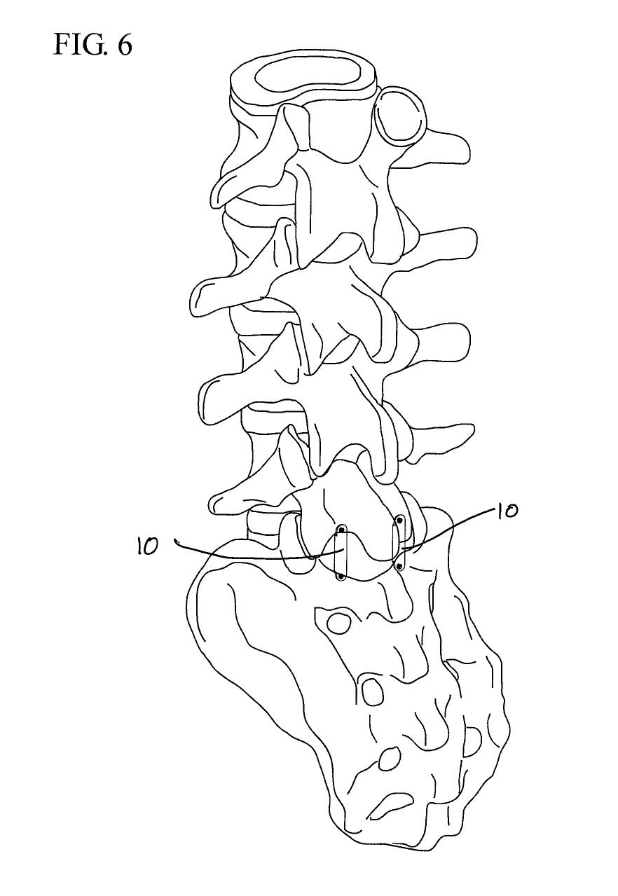

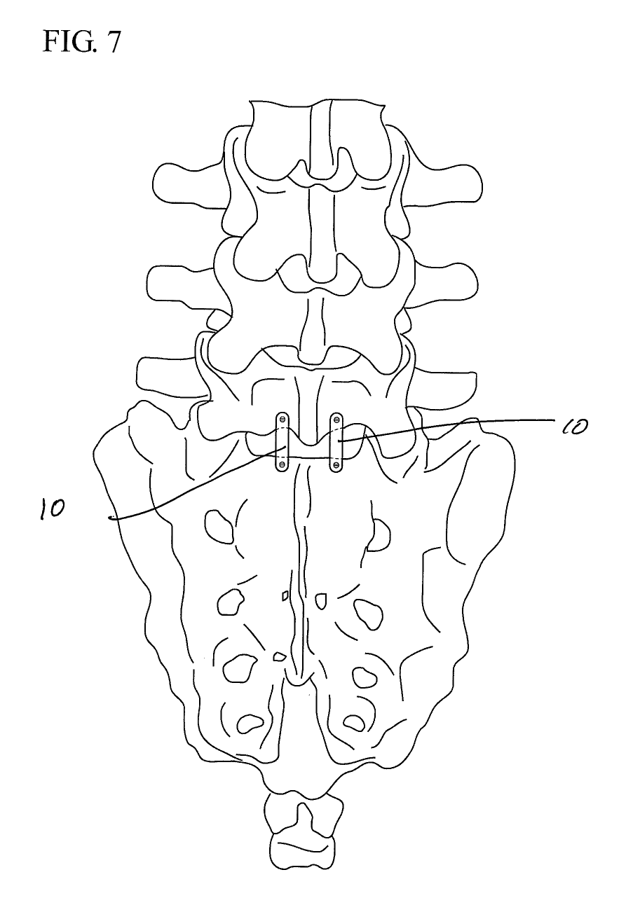

The lumbar-sacral strut is located along a portion of the spine lateral to the sagittal plane. Although a single lumbar-sacral strut may be used, two lumbar-sacral struts may be used with one such strut located on either side of the sagittal plane. See FIGS. 6 and 7.

The lumbar-sacral strut may also include a mechanism to allow the strut to be inserted in a collapsed configuration and then expanded along its longitudinal axis to a deployed configuration to maintain a desired distance between the L5 vertebra and the S1 vertebra. For example, lumbar-sacral strut 100 may be formed from a superior portion 50 and an inferior portion 70 that are movably connected to one another to allow the lumbar-sacral strut to be inserted into the patient in a collapsed configuration and then expanded once inserted to distract the space between the sacrum and the L5 vertebra. The movable connection between superior portion 50 and inferior portion may take many forms. Illustrative embodiments include a piston-type configuration. Alternatively a screw, gear or ratchet mechanism or a pulley mechanism could be used.

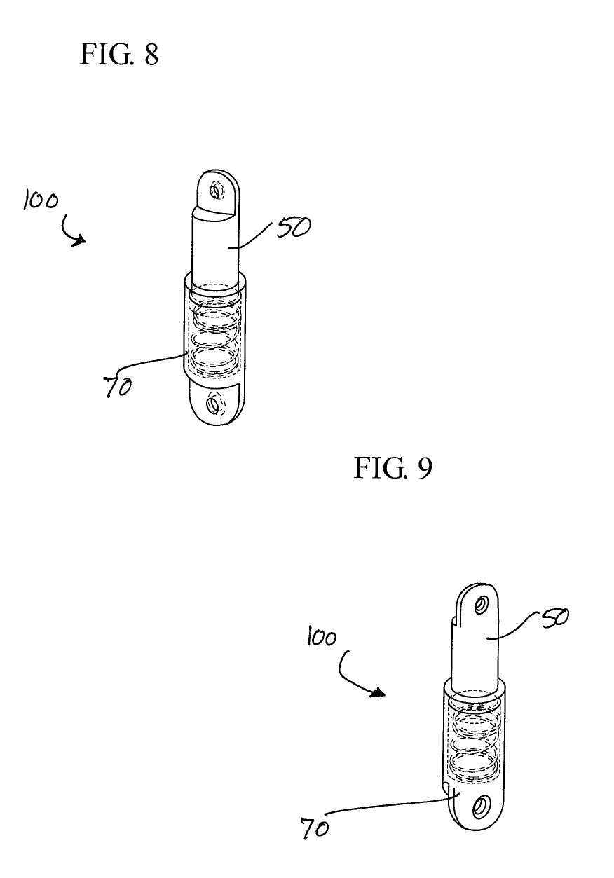

As shown, for example, in FIGS. 8 and 9 lumbar-sacral strut 100 could be formed with a piston like configuration where one of superior portion 50 or inferior portion 70 is formed as a housing for the other portion, which would travel longitudinally within the housing. In the exemplary embodiment of the FIGS., superior portion 50 is the rod portion having an inferiorly facing surface and inferior portion 70 is the cylinder having a hollow inner portion in which superior portion 50 may be movably located. A spring 60 or other biasing element, such as hydraulic fluid or a gear and ratchet mechanism, may be located in inferior portion 70 and abut the inferiorly facing surface of superior portion 50. Spring 60 would thus bias superior portion 50 and inferior portion 70 apart.

Superior portion 50 also includes a superior mounting portion which may take any of the forms previously described. As shown in FIGS. 8 and 9, both the superior mounting portion of superior portion 50 and the inferior mounting portion of inferior portion 70 include a single flange, ½ lap, rabbet-type configuration. As described in connection with the previous embodiments, the superior mounting portion of superior portion 50 and the inferior mounting portion of inferior portion 70 may take other configurations, as well, and may also include additional fixation elements.

The lumbar-sacral strut may be implanted in an open procedure. Alternatively, where the lumbar-sacral strut is formed such that it may have a collapsed configuration and an extended configuration, the lumbar-sacral strut may be implanted percutaneously.

If desired, a locking mechanism may be used to hold the superior end of the superior portion and the inferior end of the inferior portion a predetermined distance apart to maintain a desired distraction between the L5 vertebra and the S1 vertebra. For example, a set screw, locking pin or mechanical switch may be used to hold the superior end of the superior portion and the inferior end of the inferior portion a predetermined distance apart. Where a screw, gear or ratchet mechanism is used to move the lumbar-sacral strut between a collapsed configuration and an expanded configuration, the screw, gear and ratchet may be designed to lock in place when it is not actively being manipulated by the surgeon.

The lumbar-sacral strut may also be formed to allow relative anterior-posterior movement between the superior portion and the inferior portion. For example, the lumbar-sacral strut may be formed using a high compliance spring or a high compliant deformable elastic material to allow such movement. This would allow more natural movement of the spine while maintaining distraction between the L5 vertebra and sacrum.

The lumbar-sacral strut described herein can be constructed with various biocompatible materials such as, for example, titanium, titanium alloy, surgical steel, biocompatible metal alloys, stainless steel, Nitinol, plastic, polyetheretherketone (PEEK), carbon fiber, ultra-high molecular weight (UHMW) polyethylene, and other biocompatible polymeric and deformable/elastic materials.

While various embodiments of the lumbar-sacral strut have been described above, it should be understood that they have been presented by way of example only, and not limitation. Many modifications and variations will be apparent to the practitioner skilled in the art. The foregoing description of the lumbar-sacral strut is not intended to be exhaustive or to limit the scope of the invention. It is intended that the scope of the invention be defined by the following claims and their equivalents.

What is claimed is:

1. A device, comprising:
a superior body defining a superior mounting portion and having a longitudinal axis;
an inferior body defining an inferior mounting portion and having a longitudinal axis generally aligned with the longitudinal axis of the superior portion; and
wherein the superior body is disposed with respect to the inferior body so that the superior body and the inferior body are movable with respect to each other between a first configuration and a second configuration such that in the first configuration the device defines a height that is smaller than a height defined by the device when it is in the second configuration.

2. The device of claim 1 wherein the superior mounting portion includes a superior fixation element.

3. The device of claim 1 or 2 wherein the inferior mounting portion includes an inferior fixation element.

4. The device of claim 1 wherein the device is formed from a deformable material.

5. The device of claim 1 wherein the superior mounting portion defines two superiorly extending flanges and a superiorly facing surface located between the two superiorly extending flanges.

6. The device of claim 1 wherein the superior mounting portion defines a single superiorly extending flange and a superiorly facing surface.

7. The device of claim 1, 5 or 6 wherein the inferior mounting portion defines two inferiorly extending flanges and an inferiorly facing surface located between the two inferiorly extending flanges.

8. The device of claim 1, 5 or 6 wherein the inferior mounting portion defines a single inferiorly extending flange and an inferiorly facing surface.

9. The device of claim 5 wherein the superior mounting portion includes a fixation element.

10. The device of claim 6 wherein the superior mounting portion includes a fixation element.

11. The device of claim 7 wherein the superior mounting portion includes a fixation element.

12. The device of claim 8 wherein the superior mounting portion includes a fixation element.

13. The device of claim 5 wherein the inferior mounting portion includes a fixation element.

14. The device of claim 6 wherein the inferior mounting portion includes a fixation element.

15. The device of claim 7 wherein the inferior mounting portion includes a fixation element.

16. The device of claim 8 wherein the inferior mounting portion includes a fixation element.

17. A device adapted to be located between the L5 vertebra and the sacrum, comprising: a main body defining a superior mounting portion and an inferior mounting portion wherein one of the superior mounting portion and the inferior mounting portion includes a shoulder and a single flange extending away from the main body and the shoulder.

18. The device of claim 17 wherein the main body includes a superior fixation element.

19. The device of claim 17 wherein the main body includes an inferior fixation element.

20. The device of claim 17 wherein the superior mounting portion includes the single flange which extends superiorly from the shoulder and wherein the superior mounting portion includes a second superiorly extending flange extending from the shoulder and the shoulder is located between the two superiorly extending flanges.

21. The device of claim 17 wherein the superior mounting portion includes the single flange which extends superiorly from the shoulder.

22. The device of claim 17, 20 or 21 wherein the inferior mounting portion includes two inferiorly extending flanges and an inferiorly extending surface located between the two inferiorly extending flanges.

23. The device of claim 17, 20 or 21 wherein the inferior mounting portion includes the single flange which extends inferiorly from the shoulder.

24. The device of claim 17, 18 or 19 wherein the superior mounting portion includes a fixation element.

25. The device of claim 17, 18 or 19 wherein the inferior mounting portion includes a fixation element.
